(19) United States
(12) Patent Application Publication
(10) Pub. No.: US 2013/0053928 A1
(43) Pub. Date: Feb. 28, 2013

(54) DEVICE, SYSTEM AND METHOD FOR IN VIVO LIGHT THERAPY

(71) Applicants: Daniel Gat, Haifa (IL); Zvika Gilad, Haifa (IL); Elisha Rabinowitz, Haifa (IL)

(72) Inventors: Daniel Gat, Haifa (IL); Zvika Gilad, Haifa (IL); Elisha Rabinowitz, Haifa (IL)

(21) Appl. No.: 13/485,288

(22) Filed: May 31, 2012

Related U.S. Application Data
(60) Provisional application No. 61/502,906, filed on Jun. 30, 2011.
(60) Provisional application No. 61/491,605, filed on May 31, 2011.

(51) Int. Cl. A61N 5/06

(52) U.S. Cl. 607/88

(57) ABSTRACT

A swallowable in vivo therapeutic device, and a method for use of a device. The device may include a transparent case and one or more radiation sources, the radiation sources to treat the detected pathological lesions inside the gastrointestinal (GI) tract with light during the passage of the device through the GI tract. A method may include inserting into a patient a device, rotating external magnets in close proximity to the patient, thereby fully controlling the movement of the device inside the GI tract, stopping the device and activating the light radiation in areas of the pathological lesions for a predetermined period of time, and deactivating the light radiation and moving the device further through the GI tract.

### PRIOR APPLICATION DATA

The present application claims priority from prior provisional application 61/502,906 entitled “DEVICE, SYSTEM AND METHOD FOR IN-VIVO LIGHT THERAPY” and filed on Jun. 30, 2011 and claims priority from prior provisional application 61/491,605 entitled “DEVICE, SYSTEM AND METHOD FOR IN-VIVO LIGHT THERAPY” filed on May 31, 2011, each of which being incorporated by reference herein in its entirety.

### FIELD OF THE INVENTION

The present invention relates to the field of in vivo therapy. More specifically the present invention relates to a device, system and method for in vivo light therapy.

### BACKGROUND OF THE INVENTION

Light therapy involves exposure to daylight or to specific wavelengths of light-emitting lasers (LELs), light-emitting diodes (LEDs), fluorescent lamps, dichroic lamps or very bright, full-spectrum light, usually controlled with various devices. The light is administered for a prescribed amount of time and may be focused on specific body areas or lesions to treat patients. The therapeutic level of illumination has several known physiological effects.

Low level laser (or light) therapy (LLLT), a subset of light therapy, also known as cold or soft laser therapy, biostimulation, or photobiomodulation is an emerging therapeutic approach in which cells or tissue are exposed to low-levels of red and near-IR light from lasers or LEDs. The LLLT emits little or no heat, sound, or vibration. Instead of producing a thermal effect, the LLLT may act via non-thermal or photochemical reactions in the cells. It might either stimulate or (less likely) inhibit cellular function, leading to reduction of cell and tissue death, promoting healing of wound healing, increasing repair of damage to soft tissue, nerves, bone, cartilage and oral ulcerations, improvement in blood properties and blood circulation, and relief for both acute and chronic pain, edema and inflammation. Many of these use the LLLT and red light therapy in the 620-660 nm range.

The use of the LLLT in health care has been documented in the literature for more than three decades. In the LLLT the effect on a living biological system is by low energy excitation of tissues that absorb photons. This was initially thought to be a peculiar property of laser light but has recently been extended to non-coherent light produced by light-emitting diodes (LEDs). The typical power output for a low level laser device used for this therapy is in the order of 10-50 mW, and total irradiances at any point are in the order of several Joules. Energy density thereby delivered is relatively low (less than 100 mW/cm²) when compared to other forms of laser therapies (e.g., ablation, cutting). The wavelengths used for the LLLT have poor absorption in water, and thus penetrate soft and hard tissues from 3 mm to up to 15 mm. The extensive penetration of red and near-infrared light into tissues has been documented by several investigators.

The mechanisms of the LLLT are complex and may rely upon absorption of particular wavelengths light in photoreceptors within sub-cellular components, particularly the electron transport chain within the membranes of mitochondria. The absorption of light by the respiratory chain components causes a short-term activation of the respiratory chain, and oxidation of the NADH pool. This stimulation of oxidative phosphorylation leads to changes in the redox status of both the mitochondria and the cytoplasm of the cell. The electron transport chain is able to provide increased levels of promotive force to the cell, through increased supply of ATP, as well as an increase in the electrical potential of the mitochondria membrane, alkalization of the cytoplasm, and activation of nucleic acid synthesis. Because ATP is the “energy currency” for a cell, the LLLT has a potent action that results in stimulation of the normal functions of the cell.

In-vitro, animal, and clinical studies indicate that the LLLT can prevent cell apoptosis and improve cell proliferation, migration, and adhesion. The stimulatory effects of low energy lasers irradiation on cell activation have been demonstrated mainly in-vitro in a variety of cell lines. The specific actions of the LLLT are summarized in Table 1. These cellular effects support clinical applications.

In vivo treatment of internal organs with the LLLT may be achieved through the use of endoscopes and fiber optic catheters to deliver light.

TABLE 1
Effect of different wavelengths on biostimulation (Laakso
E. L. et al, “Factors affecting low level laser therapy”, Australian
Journal of Physiotherapy, 1993, 39: 95-99).

| | | Energy | | |
|---|---|---|---|---|
| Wavelength | | Density | | Effect |
| 540 nm, and | | 0-56 | J/cm² | Dose and light intensity-dependent |
| 600 to 900 nm | | | | fibroblast proliferation |
| 632.8 | nm | 2.4 | J/cm² | Vasodilation, mast cell exocytosis, |
| | | | | interstitial oedema and opening of |
| | | | | cell membrane pores |
| 632.8 | nm | 2.4 | J/cm² | Enhanced neutrophil phagocytosis |
| 632.8 | nm | 2 | J/cm² | Improved fibroblast metabolic rate |
| 632.8 and | | 0.25-4 | J/cm² | Increased keratinocyte proliferation |
| 904 nm | | | | |
| 660, 820, 870 | | 2.4 | J/cm² | Stimulation of fibroblast proliferation |
| and 880 nm | | | | by affecting macrophage responsiveness |
| 660 | nm | 2.4-9.6 | J/cm² | Enhanced macrophage responsiveness |
| | | | | and proliferation |
| 820 | nm | 2.4-7.2 | J/cm² | Increased macrophage responsiveness |
| | | | | and fibroblast proliferation |
| 830 | nm | 10 | J/cm² | Increased perfusion and angiogenesis |
| | | | | in rat skin flaps |
| 830 | nm | 10 | J/cm² | Increased phagocytic activity of |
| | | | | neutrophils |
| 904 | nm | 76.4 | J/cm² | Reduced oedema and improved rate of |
| | | | | skin wound closure in rats |

A challenge in the endoscopic application of the LLLT is the delivery and even distribution of adequate doses of light to the tissue being treated. For the treatment of some diseases in hollow substantially cylindrical organs, such as the organs of the gastrointestinal tract, it may be required to diffuse light evenly and circumferentially in a perpendicular orientation to the long axis of the fiber guide. Treatment of other pathologies may require concentration of light in a specific direction or orientation at the specific location of the pathological tissue. Another challenge associated with in vivo application of LLLT is the requirement to expose the tissue to a series of doses of light over a long period of time (e.g., days, weeks, months, or years). In some instances, each dose may require exposure for several minutes or hours. The application of a lengthy and repetitive LLLT procedure with a tethered endoscope may subject the patient to significant discomfort.

Consequently, there is a need to address the above problems associated with the use of LELs and endoscopes in the in vivo treatment of various pathologies appearing in the gastrointestinal (GI) tract with the LLLT technique.

### SUMMARY OF THE INVENTION

The aforementioned problems may be solved by using wireless in vivo therapeutic devices equipped with one or more LEDs for radiating the pathological areas.

It would be therefore beneficial to employ the wireless in vivo therapeutic device equipped with the multiple LEDs to treat various areas of the GI tract, where standard endoscopes cannot reach, to quickly deliver light to such pathologic lesions and thereby shorten duration of the treatment and relieve the patient's discomfort. In some embodiments, it would also be desirable to have full control over the movement of such in vivo therapeutic device, including maneuvering this in vivo device to a desired location and/or orientation of the device in the GI tract, and maintaining the location/orientation for as long as the light therapy of the particular location is required or needed.

Embodiments of the present invention provide a device, system and method for in vivo light therapy. The wireless in vivo therapeutic device of the present invention may be a swallowable capsule, for example, a capsule that may detect and treat pathologies in the gastrointestinal (GI) tract during its passage through the GI tract.

Light therapy within the GI tract may have an effect on bacterial mucosal colonization or eradicate GI bacteria. For example, when Small bowel bacterial overgrowth syndrome is present, light therapy may allow avoiding use of antibiotics and achieve a local effect.

In one embodiment, the in vivo therapeutic device may be an autonomous in vivo device equipped with one or more LEDs in order to radiate the desired locations inside the GI tract. The LEDs may be positioned on a printed circuit board (PCB), such as a flexible PCB, inside the transparent or partially transparent case of the device. The LEDs may radiate in a continuous, intermittent, or alternate mode. In addition, the LEDs may radiate at different wavelengths to achieve different therapeutic effects and to perform light treatment of specific pathologies in vivo, e.g., as indicated in Table 1, hereinabove.

In another embodiment, the autonomous in vivo device may comprise an additional sensor to identify the pathological area where the light treatment is desired. The sensor may be a bleeding detection sensor or pH sensor, or any other sensor, which would indicate, for example, the entrance of the in vivo device into the small bowl. The sensor could also be used to indicate entrance or exit of the in vivo device to other locations along the GI tract, e.g., the stomach or colon.

In a further embodiment, the device may radiate with a variable light intensity and at different wavelengths in a way that would be movement dependent, and optionally location dependent. The in vivo device may, for example, switch to radiating high-intensity IR light and treat the pathology lesions when it moves slowly or even stops in the areas where these pathologies are located. On the other hand, the device may switch to radiating low-intensity antibacterial UV light when it moves fast. This may help to avoid treating the areas inside the GI tract, which do not require such treatment, and depleting a power source energizing the in vivo device.

In yet another embodiment, the autonomous in vivo device may include means for slowing down the movement of the device of the invention in the predetermined areas inside the GI tract, where radiation is required, needed or desired. The device may comprise, for example, at least one compartment containing a spongy material covered by dissolvable coating. The coating is dissolved, for instance, at specific pH and/or after a predetermined period of time or as a result of another triggering event, thereby releasing the balloon-like sponge or sponges. Theses sponges once expanded significantly increase the total volume of the device, and hence, slow its motion. Other types of expanding materials other than sponges may be used.

In yet a further embodiment, the in vivo therapeutic device may be fully controlled including being maneuvered to a desired location and/or orientation of the device in the GI tract, and being maintained in that location/orientation for as long as the light therapy of the particular location is required or needed. This in vivo device may include a permanent magnets assembly for interacting with external magnetic fields for generating forces for steering the device. In addition, this in vivo therapeutic device may include a multilayered imaging and sensing printed circuit board for sensing the current location and orientation of the in vivo device, and for transmitting corresponding location and orientation data to an external system that generates the external magnetic fields.

In still another embodiment, the in vivo therapeutic device of an embodiment of the invention is essentially floatable or has neutral or close to neutral buoyancy, and may include additional means for docking the device to the GI tract walls at or in proximity of the desired location, where the light therapy is required or needed. In one embodiment, the in vivo device may include a detachable therapeutic head. This detachable head may be equipped with special means for docking the head to the GI tract walls and with one or more LEDs in order to radiate the desired locations inside the GI tract. Once such an in vivo device reaches the specific location inside the GI tract, where the light therapy is required or needed, the detachable head may be detached from the body of the device and “hooked” onto the tissue of the GI tract walls. The LEDs of the detachable head may then radiate the pathological lesions in the tissues of the GI tract walls for either predetermined periods of time or until the batteries are depleted.

The details of one or more embodiments are set forth in the accompanying figures and the description below. Other features, objects and advantages of the described techniques will be apparent from the description and drawings and from the claims.

### BRIEF DESCRIPTION OF THE DRAWINGS

The present invention will be understood and appreciated more fully from the following detailed description taken in conjunction with the appended figures. Various exemplary embodiments are illustrated in the accompanying figures with the intent that these examples not be restrictive. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements. Of the accompanying figures:

FIGS. 1A-1B are perspective and cross-sectional views, respectively, of an autonomous in vivo therapeutic device for light therapy according to an embodiment of the invention;

FIG. 2 is a graphic representation of an autonomous in vivo therapeutic device for light therapy comprising a bleeding detection sensing head according to an embodiment of the invention;

FIG. 3A is a schematic view of an autonomous in vivo therapeutic device for light therapy comprising compartments containing a balloon-like expandable material covered by a dissolvable coating according to an embodiment of the invention;

FIG. 3B is a schematic view of the autonomous in vivo therapeutic device shown in FIG. 3A after the coating is dissolved, thereby releasing the balloon-like expandable material according to an embodiment of the invention;

FIGS. 4A-4B are perspective and cross-sectional views, respectively, of a fully controllable and maneuverable in vivo therapeutic device for light therapy according to an embodiment of the invention;

FIG. 5 is a schematic view of a detachable head docked to the GI tract walls according to an embodiment of the invention; and

FIG. 6 is a diagram describing the method of using a fully controllable and maneuverable in vivo device for light therapy according to an embodiment of the invention.

### DETAILED DESCRIPTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

The in vivo device of an embodiment of the invention may typically be autonomous and typically self-contained. For example, a device according to some embodiments may be a capsule or other unit where all the components are substantially contained within a case, housing or shell, and where, for example, the device does not require wires or cables in order to receive power or transmit information.

According to particular embodiments of the invention, the in vivo device is essentially floatable or has a neutral or near neutral buoyancy in water or in other liquids that may be present within body lumens. According to some embodiments, the in vivo device may be designed to access pathologic lesions or lumen wall tissue in nearly every region of the GI tract, e.g., the colon and biliary tree. In some embodiments, the in vivo device may be designed to illuminate the pathological areas alone and to spare or skip healthy areas. The in vivo device may be designed to access and treat difficult to reach areas, where standard tethered endoscopes cannot reach or cannot easily reach. The in vivo device may quicken the delivery of energy to the desired regions and thereby result in a short, easy and painless administration and treatment.

Some embodiments of the present invention are directed to a swallowable in vivo therapeutic capsule that may be used for treating the pathological areas inside the GI tract with light.

In some embodiments, illumination or radiation sources used within the in vivo device may include, for example, light emitting diodes (LEDs), incandescent sources, or other suitable light sources that may enable in vivo radiation, and may include devices providing electromagnetic radiation within the visible spectrum, outside of the visible spectrum, and further a combination of visible and non-visible electromagnetic radiation.

According to some embodiments, the in vivo device, which passes through the GI tract, may additionally include one or more imaging sensors, or imagers. The imagers may capture images of the interior of the GI tract, record and transmit in vivo data, such as, for example, from the entire length of the GI tract, to a receiving and/or processing unit. In some embodiments, the processing unit may be external to the in vivo device, and may be part of an external receiving unit, though in other embodiments, the processing unit may be housed within the in vivo device. Other in vivo devices may alternatively or additionally include a medication container and means for administering medication to within the GI tract. Other in vivo devices may include means for performing treatment operations in vivo.

The in vivo therapeutic device may communicate with an external receiving and display system to provide display of data, control, or other functions. Power may be provided, for example, by an internal battery or a wireless receiving system. Other embodiments may have other configurations and capabilities. Components in some cases may be distributed over multiple sites or units and control information may be received from an external source. The devices according to embodiments of the present invention, their principles of operation, the inner structure, as well as receiving, storage, processing and/or display systems suitable for use with embodiments of the present invention may be similar to embodiments described in PCT Application Publication No. WO 01/65995,

U.S. Pat. No. 7,009,634, which is assigned to the common assignee of the present invention and which is hereby incorporated by reference in its entirety. Components of the device according to embodiments of the present invention may be similar to components used in the PillCam® capsule endoscopy system commercially available from the common assignee of the present invention. Of course, devices, systems, structures, functionalities and methods as described herein may have other configurations, sets of components and processes, etc.

It should be noted that while a device, system and method in accordance with some embodiments of the invention may be used, for example, in a human body, the invention is not limited in this respect. For example, some embodiments of the invention may be used in conjunction with or inserted into a non-human body, e.g., a dog, a cat, a rat, a cow, or other animals, pets, laboratory animals, etc.

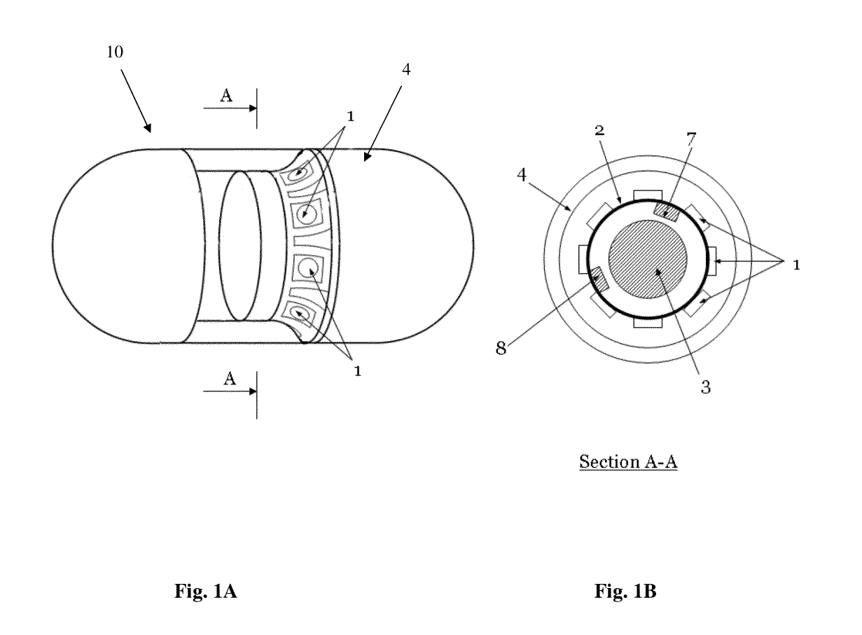

Reference is now made to FIGS. 1A and 1B, which illustrate the perspective and cross-sectional views, respectively, of an autonomous in vivo therapeutic device 10 for light therapy. This in vivo therapeutic device 10 may be fully autonomous and may be equipped with one or more illumination sources 1, e.g., LEDs, in order to radiate the desired locations inside the GI tract. LEDs 1 may be positioned on PCB 2, such as a fully flexible PCB, inside transparent or partially transparent case 4 of device 10. LEDs 1 may radiate in a continuous, intermittent, or alternate mode through the transparent portions of case 4. In addition, LEDs 1 may radiate at different wavelengths to achieve different therapeutic effects and to perform light treatment of specific pathologies in vivo, e.g., as indicated in Table 1, hereinabove.

As shown in FIG. 1B, in vivo device 10 may comprise, for example, power source 3, such as batteries, microcontroller 7 and switch 8, all located within case 4. Microcontroller 7 may set a timer for a predetermined period of time and activate illumination sources 1 using switch 8 whenever the device reaches particular areas of the GI tract where pathological lesions are detected and light therapy is desired or needed. After the light therapy is completed, switch 8 may turn off LEDs 1, thereby deactivating device 10.

Multiple LEDs 1 may be placed around the perimeter of case 4 or alternatively, on a base located closer to the center of device 10, for example, on PCB 2, as shown in FIG. 1B, in order to distance LEDs 1 from the surface of case 4 and from the illuminated tissue and thus achieve a wider illumination area. The base onto which LEDs 1 are positioned may optionally comprise one or more components, for example, microcontroller 7 and switch 8. Other arrangements are also possible, for example several LEDs 1 may be angled relative to a specific axis in order to create different angles of radiation and in order to radiate, for example, a selected area of desired locations inside the GI tract. Other designs, elements, and structures may be used in addition to and/or in place of the aforementioned components. The width of the base structure supporting LEDs 1, e.g., PCB 2, may be controlled.

In vivo device 10 as depicted in FIG. 1A is generally capsule shaped, and may be easily swallowed and passively passed through the entire GI tract, pushed along, for example, by natural peristalsis. Nonetheless, it should be noted that device 10 may be of any shape and size suitable for being inserted into (e.g., by swallowing or by a delivery device) and passing through a body lumen or cavity, such as spherical, oval, cylindrical, etc., or other suitable shapes. Furthermore, in vivo device 10 may comprise some additional components, which may be attached or affixed onto an instrument that is inserted into body lumens and cavities, such as, for example, on an endoscope, laparoscope, needle, catheter, etc.

Case 4 of device 10 may be made transparent in order to allow a full 360° radiation of the inner portions of the GI tract. Multiple LEDs 1 may be assembled on a strip and positioned and shaped according to the shape of in vivo device 10 and according to specific light radiating requirements, so as to avoid phenomena (e.g., backscatter) that may be associated with illuminating from within a window.

The radiation strip including LEDs 1 may be flexible and may, for example, bend in a range of degrees such that it may conform to the shape of case 4 upon insertion of the strip, e.g., PCB 2, into case 4 so as to enable, for example, an outwards radiation at different angles. The radiation angle may be determined by the shape of case 4. PCB 2 may further include contact points to connect additional components.

In some embodiments, device 10 may be equipped with different LEDs 1, which radiate at different wavelengths, in order to achieve different therapeutic effects and to perform light treatment of specific pathologies in vivo, for example, as indicated in Table 1, hereinabove. Other wavelengths may be used.

In addition, the device may radiate with a variable light intensity at different wavelengths in a way that would be movement dependent, and optionally location dependent. The autonomous in vivo device may, for example, switch to radiating high-intensity IR light and treat the pathology lesions when it moves slowly or even stops in the areas where these pathologies are located. The device may switch to radiating low-intensity antibacterial UV light when it moves fast and/or when it is no longer near the pathological area. This may help to avoid treating the areas inside the GI tract, which do not require such light therapy treatment, and further avoid early depletion of a power source energizing in vivo device 10.

In some embodiments, in vivo therapeutic device 10 may operate in an automatic mode, which is referred to herein as an adaptive intensity mode, in which in vivo device 10 may transition from a first operational mode, e.g., from a low intensity mode, to a second operational mode, e.g., to a high intensity mode, and vice versa, contingent on estimated movements of the in vivo device. Device 10 may alternatively or additionally transit from the first operational mode to the second operational mode, and vice versa, contingent on the location of the in vivo device in the GI tract. While two modes of relative intensities are discussed, other numbers of modes of relative intensities and predefined wavelengths may be used.

The power condition of the charge storing device may be used to enable and disable the adaptive intensity mode. For example, if the power level of the charge storing device reaches a certain level, it may be decided to disable the adaptive intensity mode and to enable the in vivo device to operate only in one mode, for example only in the low intensity mode, in order to preserve energy.

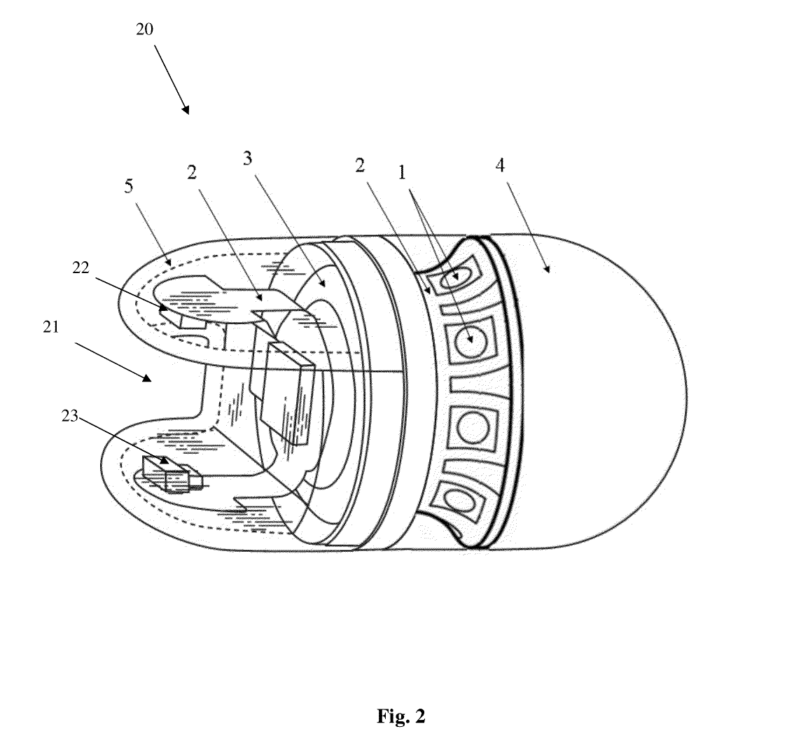

Reference is now made to FIG. 2, which shows a graphic representation of an autonomous in vivo therapeutic device for light therapy comprising a bleeding detection sensing head. As shown in FIG. 2, device 20 may comprise multiple LEDs 1, which are positioned on stepped PCB 2 or on the radiation strip, power source 3, e.g., batteries and bleeding detection sensing head 5. Bleeding detection sensor 5 enables identifying the pathological area inside the GI tract where the light treatment is desired or needed. Bleeding detection sensing head 5 is similar to the sensing head of the bleeding sensing capsule, which is disclosed in PCT Application Publication No. WO 2010/086859 assigned to the common assignees of the present invention and incorporated herein by reference in its entirety. The bleeding detection sensing head 5 may comprise gap 21, which is substantially in constant contact with in vivo fluids, such that in vivo fluids freely flow in and out of gap 21. Several LEDs 22, which may be different from LEDs 1, may be encapsulated in sensing head 5. LEDs 22 may be positioned on one side of gap 21, illuminating at different wavelengths, while on the opposite side of gap 21 there may be at least one light detector photodiode 23. The light detector photodiode is typically positioned such that it is facing illuminating LEDs 22, while gap 21 is placed in between the LEDs 22 and light detector photodiode 23. Light illuminated by the LEDs 22 passes through the in vivo fluids and onto light detector photodiode 23. Some of light may be absorbed by the in vivo fluids, some may be reflected, and some may be transmitted to light detector photodiode 23, which may then transmit signals, created in response to the detected light, to an external receiver (not shown). A processor, external to the device, may process the signal sent by light detector 23 and create an absorption or transmission spectra of the in vivo fluids. By comparing the signals to a reference transmission spectrum of bile and to a reference transmission spectrum of blood, it may be determined whether bile, blood or both are present in vivo, and in what concentration, such that a conclusion may be made regarding presence of pathologies in vivo. In other embodiments, instead of comparing transmission or absorption spectra, a comparison between discrete signals detected by light detector photodiode 23 and a predetermined threshold may be done.

In vivo device 20 shown in FIG. 2 may alternatively include a pH detector (not shown), which may be located at the current location of sensing head 5. The pH detector may continuously detect pH levels of the body lumen liquid inside the GI tract. Such a pH detector may comprise two electrodes and an electrical circuit, and may transmit the detected pH levels to a receiver external to a patient's body. Since, in different areas of the GI tract, there are different pH levels, the detected pH level may indicate the location of various pathological lesions inside the GI tract where light therapy is needed. Optionally, device 20 may comprise both a bleeding sensing head and a pH detector such to combine the two methods, e.g., use detection of both the absorption or transmission spectra and the pH level in order to indicate location of pathological lesions.

According to some embodiments, the in vivo therapeutic device may include means for slowing down its movement in predetermined areas inside the GI tract, where radiation is required or needed. The device may be configured to change its shape or geometry when entering certain parts of the GI tract, such as the large intestine, so that it may be better adjusted to movement through a large body lumen.

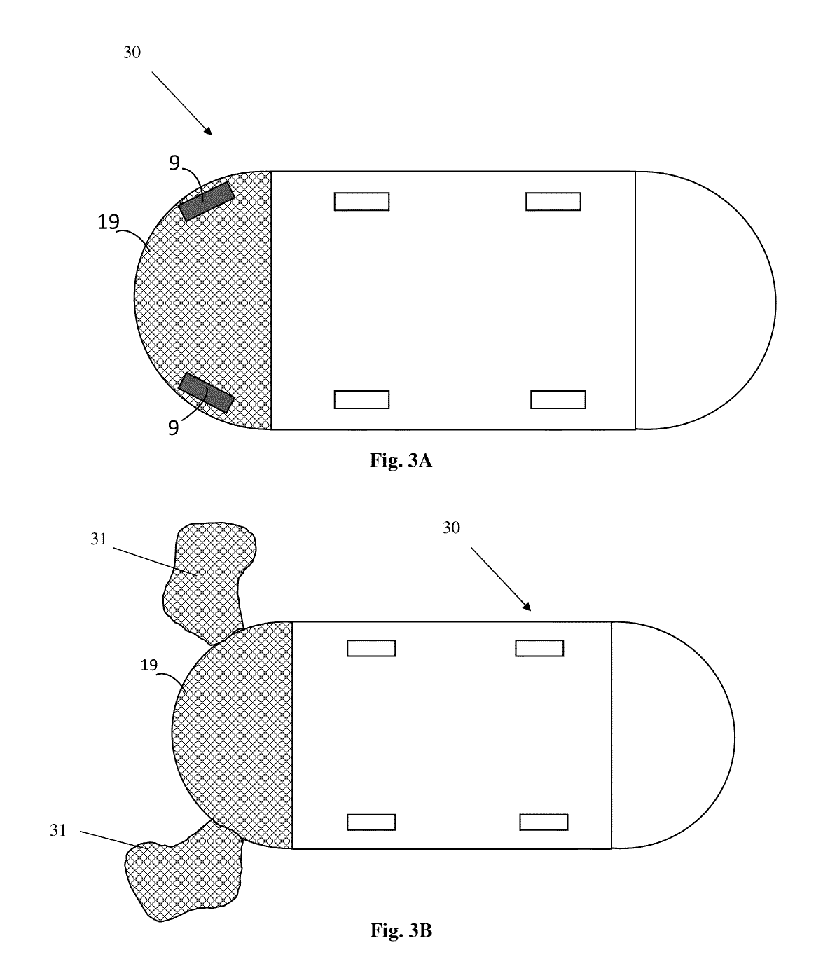

Reference is now made to FIG. 3A, which illustrates a schematic view of an autonomous in vivo therapeutic device for light therapy, comprising compartments containing a balloon-like expandable material covered by a dissolvable coating. As shown in FIG. 3A, device 30 may comprise at least one compartment containing expandable material 10, such as a spongy material. Expandable material 10 may be covered by dissolvable coating 9. The coating may be dissolvable at specific pH or alternatively, after a predetermined period of time has elapsed. As shown in FIG. 3B, once certain pH is reached in the body lumen liquid of the GI tract, coating 9 may dissolve, thereby releasing the expandable material 10 from within the compartment, such that the expandable material may acquire a balloon-like shape 31. Once expanded, theses balloon-like sponges 31, may significantly increase the total volume of device 30, and hence, slow its motion. Spongy material 10 may be substituted by any pliant or soft material, such as, for example, rubber or silicone, and may be of any shape that is useful in slowing the motion of device 30 near the pathological areas inside the GI tract, and further positioning device 30 to provide the wide field of radiation and hence, enable efficient light therapy of the pathologic lesions. For example, the released balloon-like sponges 31 may be in the form of cone-shaped appendages or wing-like appendages expanded in a plane perpendicular to the longitudinal axis of the device. In some embodiments, the plane at which the appendages expand is also perpendicular to the general direction of motion of the device.

Thus, autonomous in vivo therapeutic device 30 of the invention may initially be completely inactive and may move freely through the GI tract by using a peristaltic force until it reaches specific areas, where pathologies are located. Device 30 may then be activated, made to change an operational mode, may be slowed down by releasing the balloon-like sponges 31 and/or stopped completely at one or more specific locations inside the GI tract in order to treat pathologies located therein with light.

Moving an autonomous device in vivo by using a peristaltic force has a few drawbacks. For example, the in vivo device may get stuck somewhere in the GI tract for an unknown period of time; the device may radiate in one direction while a nearby area, which requires light treatment, is not radiated sufficiently if at all. Therefore, another embodiment of the invention is a fully controlled in vivo therapeutic device, which may be maneuvered to a desired location and/or orientated in the GI tract maintaining the location/orientation for as long as the light therapy of a particular pathology is required or needed.

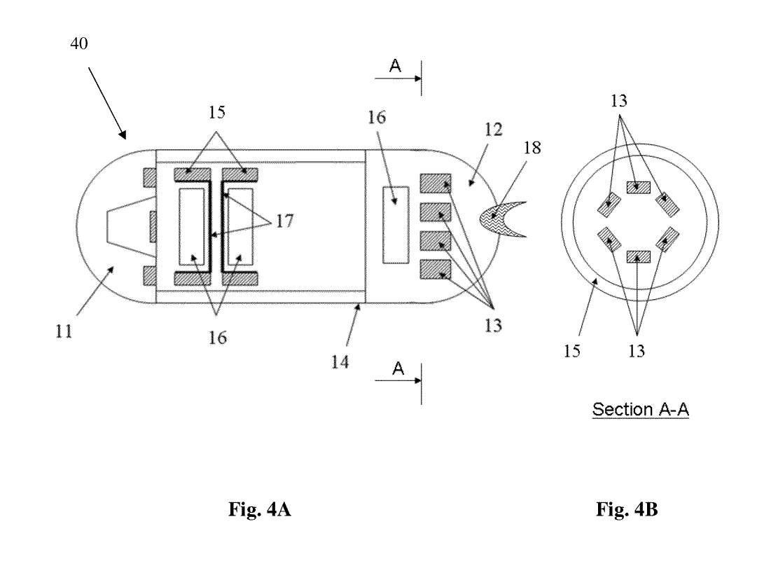

Reference is now made to FIGS. 4A and 4B which illustrate the perspective and cross-sectional views, respectively, of a fully controllable and maneuverable in vivo therapeutic device for light therapy. Device 40 may comprise an imaging head 11 and a detachable therapeutic head 12 located within transparent case 14. In vivo device 40 may include a permanent magnets assembly 15 for interacting with external magnetic fields for generating forces for steering the device.

The fully controllable in vivo therapeutic device 40 shown in FIG. 4 may comprise a multilayered imaging and sensing printed circuit board for sensing the current location and orientation of in vivo device 40 inside the GI tract, and transmitting the corresponding location and orientation data to an external system that generates the external magnetic fields. Such controllable therapeutic in vivo device may comprise one or more imagers for capturing images of the interior of the GI tract.

Imaging head 11 of device 40 may comprise one or more radiation sources, such as LEDs or other suitable radiation sources, and lenses placed inside the transparent convex (e.g., dome) optical window 11 of device 40, on a PCB or other suitable support.

The PCB may optionally comprise one or more components, for example, conductive rings, and/or conductive strips. The PCB may also comprise other components of device 40 such as an antenna typically associated with a transmitter for transmitting images from the optional imager.

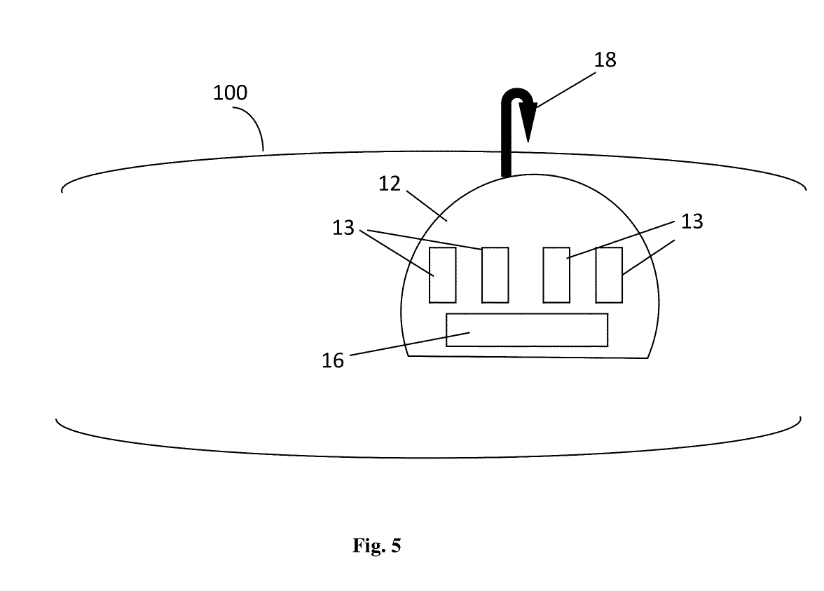

Fully controllable in vivo therapeutic device 40 may additionally comprise a detachable therapeutic head 12. Detachable head 12 may be equipped with special means 18 for docking the head to the GI tract walls. Detachable head 12 may further comprise one or more LEDs 13 in order to radiate the desired locations inside the GI tract. As shown in FIG. 5, once in vivo device 40 reaches the specific location inside the GI tract, where light therapy is required or needed, detachable head 12 may detached from the body of device 40 and “hooked” or otherwise attached to the tissue of the GI tract walls 100. LEDs 13 of detachable head 12 may then radiate the pathological lesions in the tissues of the GI tract walls for either predetermined period of time or until the batteries are depleted.

Means 18 for docking detachable head 12 to the GI tract walls at the desired location, where the light therapy is required or needed, may be, for example, one or more anchors. The anchors may have an arrowhead capable of piercing the GI tract walls and may have any size and shape which may hold detachable head 12 in place. Such anchors may be formed of any biodegradable material strong enough to hold the torch head in place but which may be soluble in the liquid environment of the GI tract. Suitable materials are, for example, caramel, biodegradable plastic resins or starches, such as gelatin, or wax. After a predetermined period of time, once the light therapy is completed, at least the arrowhead of the anchors may be dissolved, thereby releasing detachable head 12 into the GI tract. Alternatively, the docking mechanism may include biocompatible adhesive, vacuum, and/or biodegradable or non-degradable hooks or pins.

Multiple LEDs 13, which may be encapsulated inside detachable head 12, may be positioned on the radiation strip or on the stepped PCB, in order to distance them from the surface of the device's case and thus achieve a wider field of radiation. The width of the base structure supporting radiation sources 13 may be controlled. The radiation strip including radiation sources 13 may, for example, bend in a range of degrees upon inserting the PCB into detachable head 12 so as to enable, for example, an outwards radiation at different angles. The radiation angle may be determined by the shape of detachable head 12. Furthermore, LEDs 13 may be positioned at different angels relative to the longitudinal axis of the device in order to radiate, for example, a selected area or the desired locations inside the GI tract. Other arrangements may also be possible, for example the LEDs may be angled relative to a different axis. Other designs, components, elements, and structures may be used in addition to and/or in place of rings, steps, etc. LEDs 13 may radiate at different wavelengths to achieve different therapeutic effects and to perform light treatment of specific pathologies in vivo, as indicated in Table 1, hereinabove.

The in vivo therapeutic device shown in FIG. 4 may comprise batteries 16, cylindrically shaped permanent magnets 15 to transform an external electromagnetic field into a maneuvering force in order to rotate and steer device 40, eddy current manifold 17 to restrain the movement of the device, and cylindrically shaped electromagnetic field sensing coil(s) (not shown) in order to sense localization signals.

Device 40 may optionally include an imager for capturing images of the GI tract, a lens holder and also a transmitter for transmitting the images captured by the imager. Typically, the imager, lens holder may be located behind optical window 11.

In some embodiments, the imager may be based on real time image processing that will identify lesions or diseased segment. Following identification of the lesions, the operator may activate a specific wavelength of LEDs 13 for appropriate light therapy, which depends on the type of lesion.

Alternatively, a predetermined area of pathology inside the GI tract to be treated with light may be marked, for example, by a color mark, an RFID (radio frequency identification) tag implanted or fixed at or before said area, or by other methods. Such marking may be carried out, for example, using an endoscope or maneuvered capsule endoscope. The in vivo therapeutic device of the invention may be equipped with a sensor to identify the mark and the device may then be slowed, stopped, activated, and/or made to change the operation mode near, at or after the marked region. Such sensor may be, for example, an imager or light-sensor and an image analysis unit capable of detecting a color mark or a scanner capable of detecting the proximity of an RFID tag. For example, if the sensor is an RFID scanner or any other sensor that is not based on detection of a color mark, the in vivo device may be free of the imaging components.

A system for in vivo light therapy according to embodiments of the invention may comprise device 40, and an external rotatable magnets assembly for steering the internal magnets of device 40 and thereby fully controlling its movement inside the GI tract.

According to some embodiments, a method of using an in vivo therapeutic device may comprise a series of multiple ingestions, e.g., ingesting 30 swallowable in vivo devices, one capsule per day. This session of ingestions may achieve the desired accumulating clinical effect of light therapy. Such a session of multiple ingestions may comprise more than one type of swallowable in vivo device, e.g., capsule shaped. For example, each device may comprise a different combination of LEDs or wavelengths.

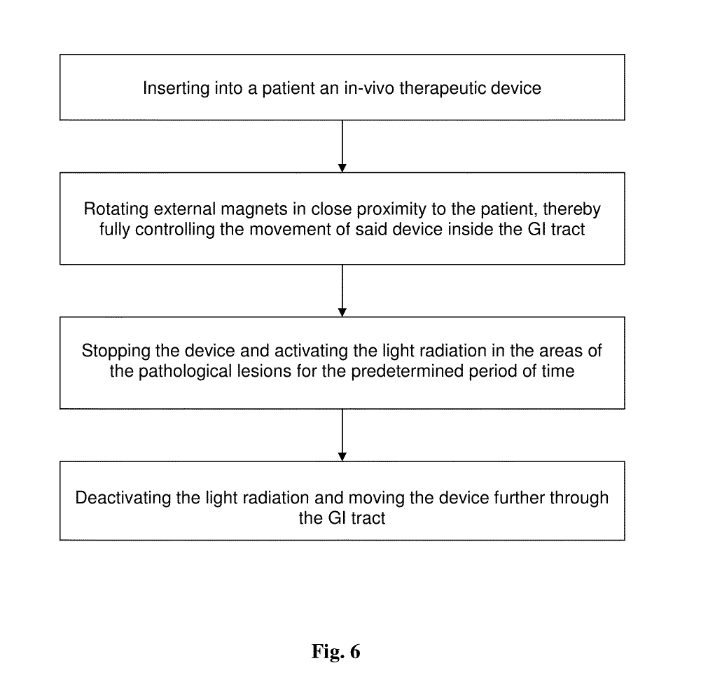

Reference is now made to FIG. 6 showing a diagram describing the method of using a fully controllable and maneuverable in vivo device for light therapy. The method for in vivo light therapy according to an embodiment may comprise in one embodiment:
inserting into a patient a device of the present invention, as described hereinabove;
rotating external magnets in close proximity to the patient, thereby fully controlling the movement of said device inside the GI tract;
stopping the device and activating the light radiation in the areas of the pathological lesions for the predetermined period of time; and after that
deactivating the light radiation and moving the device further through the GI tract.

It will be appreciated that the present invention is not limited to what has been particularly shown and described hereinabove. Rather the scope of the present invention is defined only by the claims which follow.

1. A swallowable in vivo therapeutic device for in vivo light therapy comprising a transparent case and one or more radiation sources, said radiation sources to treat the detected pathological lesions inside the gastrointestinal (GI) tract with light during the passage of the device through the GI tract.

2. The device according to claim 1, wherein said device is autonomous.

3. The device according to claim 1, wherein said radiation sources are selected from a group consisting of light emitting diodes (LEDs), incandescent sources, or any other suitable light sources that may enable in vivo radiation.

4. The device according to claim 1, wherein said radiation sources provide an electromagnetic radiation selected from a group consisting of electromagnetic radiation within the visible spectrum, outside of the visible spectrum, or a combination of visible and non-visible radiation.

5. The device according to claim 1, wherein said radiation sources may radiate in a continuous or alternate mode.

6. The device according to claim 1, wherein said radiation sources radiate at different wavelengths to achieve different therapeutic effects and to perform light treatment of specific pathologies in vivo.

7. The device according to claim 1, where said device has adaptive intensity mode.

8. The device according to claim 2, said device comprising a power source, a microcontroller and an RF switch.

9. The device according to claim 1, wherein said device comprises one or more sensors to identify the pathological area where the light treatment is desired.

10. The device according to claim 10, wherein said sensor is a bleeding detection sensor or a pH sensor.

11. The device according to claim 10, wherein said bleeding detection sensor comprises:
a gap in the transparent case of the device, wherein in vivo fluids may flow through said gap;
illumination sources on one side of the gap, wherein each illumination source illuminates the in vivo fluids at a different narrow band illumination; and
at least one light detector positioned at the opposite side of the gap and facing the illumination sources, for detecting light which passes through the in vivo fluids.

12. The device according to claim 1, wherein said device is essentially floatable.

13. The device according to claim 1, wherein said device comprises at least two compartments containing a appendages made of spongy, pliant or soft material covered by a dissolvable coating.

14. The device according to claim 13, wherein said dissolvable coating is configured to dissolve after a predetermined period of time or at a specific pH, thereby releasing the appendages.

15. The device according to claim 1, wherein said device is a fully controllable and maneuverable in vivo device.

16. The device according to claim 1, wherein said device comprises a permanent magnets assembly for interacting with external magnetic fields for generating forces steering the device and maneuvering it to a desired location and/or orientating it inside the GI tract, and maintaining the location/orientation for as long as the light therapy of a particular pathological lesion is required.

17. The device according to claim 1, wherein said device comprises conductive rings, and/or conductive steps.

18. The device according to claim 1, wherein said device comprises an antenna and a transmitter for transmitting images captured by the imager.

19. A method for in vivo light therapy comprising: inserting into a patient a device according to claim 1;
rotating external magnets in close proximity to the patient, thereby fully controlling the movement of said device inside the GI tract;
stopping the device and activating the light radiation in areas of the pathological lesions for a predetermined period of time; and
deactivating the light radiation and moving the device further through the GI tract.

20. A system for in vivo light therapy comprising:
the device according to claim 1; and
an external rotatable magnets assembly for steering the internal magnets of said device and thereby fully controlling its movement inside the GI tract.
